(19) United States
(12) Patent Application Publication
(10) Pub. No.: US 2017/0273911 A1
(43) Pub. Date: Sep. 28, 2017

(54) INJECTABLE MICROSPHERES

(71) Applicant: Boston Scientific SciMed Inc., Maple Grove, MN (US)

(72) Inventors: Michael Walsh, Galway (IE); Emma Jane Mooney, Co. Galway (IE); Javier Palomar-Moreno, Galway (IE); Damien Vincent Nolan, Galway (IE); Salman Musani, Galway (IE); Marie Turkington, Co. Mayo (IE)

(21) Appl. No.: 15/453,406

(22) Filed: Mar. 8, 2017

Related U.S. Application Data
(60) Provisional application No. 62/312,216, filed on Mar. 23, 2016.

(51) Int. Cl. A61K 9/50; A61M 25/06; A61K 35/12; A61M 25/00; A61K 45/06; A61K 9/00

(52) U.S. Cl. CPC A61K 9/5031; A61K 9/5073; A61K 9/5089; A61K 45/06; A61K 9/0019; A61K 35/12; A61M 25/0043; A61M 25/0606; A61M 2210/12

(57) ABSTRACT

An injectable composition for intravascular delivery of a therapeutic agent includes one or more first microspheres containing a first therapeutic agent, one or more second microspheres containing a second therapeutic agent, and a liquid carrier. The first microspheres includes a wall comprising a biodegradable polymer that encapsulates the first therapeutic agent and the second microspheres includes a wall comprising the biodegradable polymer that encapsulates the second therapeutic agent.

This application claims the benefit of U.S. Provisional Application No. 62/312,216, filed Mar. 23, 2016, the contents of which are herein incorporated by reference.

### TECHNICAL FIELD

This document relates to systems, devices, and methods relating to injectable microspheres for delivering a therapeutic agent within a patient's vasculature.

BACKGROUND

Critical limb ischemia (CLI) is associated with severe obstruction of blood flow to a person's extremities (e.g., arms, legs, or feet) that has potential to eventually lead to limb loss. The symptoms associated with CLI can include pain in the foot at rest, non-healing ulcers, limb/digital gangrene, and delayed wound healing. An estimated 160,000 to 180,000 amputations are performed annually in the United States due to CLI. The rate of lower limb amputation in the United States has doubled since 1985 with a 4- to 5-fold increase in those over the age of 80. Fewer than half of all CLI patients may achieve full mobility after an amputation, and only one in four above-the-knee amputees will ever wear a prosthesis. The estimated cost of treating CLI is currently about 10 to 20 billion dollars per year in the US alone.

The quality of life for those with CLI can be extremely poor and reported to be similar to that of patients with end stage malignancy. Most patients with CLI may undergo repeat hospitalizations and surgical/endovascular procedures in an effort to preserve the affected limb(s). In certain circumstances, limb salvage efforts are not effective enough to reverse ischemia, and despite multiple attempts at revascularization, one or more wounds may fail to heal properly. In addition, many patients may not be eligible candidates for traditional forms of revascularization due to occluded or diffusely diseased distal vessels. Accordingly, there is a need in the art for therapies and devices that can treat critically ischemic limbs.

### SUMMARY

Disclosed herein are various embodiments of devices, systems, and methods relating to injectable compositions that include microspheres for delivering a therapeutic agent.

In a first aspect, an injectable composition for intravascular delivery of a therapeutic agent includes one or more first microspheres containing a first therapeutic agent, one or more second microspheres containing a second therapeutic agent, and a liquid carrier. Each first microsphere can include a wall containing a biodegradable polymer that encapsulates the first therapeutic agent. Each second microsphere can include a wall containing the biodegradable polymer that encapsulates the second therapeutic agent.

In some cases, the first therapeutic agent, the second therapeutic agent, or both, can be selected from a group consisting of stem cells, adenoviruses, chemotherapeutic agents, immunosuppressant, proteins, nucleic acids, or a combination thereof. Preferably, the injectable composition provided herein can include the first and second microspheres in an amount ranging from 10% to 50% by weight of the injectable composition and the carrier in an amount ranging from 90% to 50% by weight of the injectable composition. In some cases, the first and second microspheres are included in the injectable composition in an amount ranging from 35% to 45% by weight of the injectable composition and the carrier in an amount ranging from 65% to 55% by weight of the injectable composition. Preferably, the first microspheres, the second microspheres, or both, are suspended in the liquid carrier. In some cases, one or more first microspheres are disposed within at least one second microsphere. In some cases, the liquid carrier can include purified water, distilled water, saline solution, or a serum. In some cases, the wall of the first microspheres has a faster degradation rate than the wall of the second microspheres. In some cases, the average wall thickness of the first microspheres can be greater than an average wall thickness of the second microspheres. In some cases, the walls of the first microspheres, the second microspheres, or both, can include a nonporous polymer layer. Preferably, the first microspheres can further encapsulate at least a portion of the liquid carrier such that the first therapeutic agent is suspended within the liquid carrier inside the first microspheres.

Preferably, the injectable composition provided herein can include the first microspheres, the second microspheres, or both, that have diameters ranging from about 0.2 millimeters to about 5.0 millimeters. In some cases, the biodegradable polymer can include polylactic acid (PLA), polyglycolic acid (PGA), a copolymer of PLA and PGA, poly-L-lactide (PLLA), poly-D,L-lactide (PDLA), poly-capralactone (PCL), or a combination thereof.

In a second aspect, a system for intravascular delivery of an injectable composition contains a catheter, a source of the injectable composition according to any one of the preceding claims in fluid communication with the lumen of the catheter, and a transporting element for transporting the injectable composition through the catheter. Preferably, the catheter can include a proximal end, a distal end, and an elongate tubular shaft defining a lumen.

In some cases, the transporting element can include an injector for applying an injection pressure to transport the injectable composition through the catheter. The transporting element can optionally include a dispensing device that includes a cup-shaped tip for contacting at least a portion of one or more microspheres and pushing the one or more microspheres through the catheter.

In a third aspect, a method of manufacturing an injectable composition includes forming a polymer membrane including PLGA or PLLA. The method provided herein can include adding a therapeutic agent and encapsulating the therapeutic agent within the polymer membrane.

Preferably, the forming and encapsulating steps can include adding an immiscible therapeutic agent into a polymeric solution including dimethylformamide and PLGA, or PLLA. In some cases, the forming step can include extruding a thin film or microtube or micropellet including PLGA or PLLA. In some cases, the encapsulating step can include injecting the therapeutic agent into a film such that the film stretches to form a microsphere.

While multiple embodiments are disclosed herein, still other embodiments of systems, devices, and/or methods will become apparent to those skilled in the art from the following detailed description, which shows and describes illustrative embodiments of systems, devices, and/or methods provided herein. Accordingly, the drawings and detailed description are to be regarded as illustrative in nature and not restrictive.

### BRIEF DESCRIPTION OF THE DRAWINGS

FIG. 1 is a schematic view of an exemplary intravascular delivery system for delivering an injectable composition provided herein within a patient's peripheral vasculature.

FIG. 2 is a graph illustratively comparing two different exemplary microspheres (e.g., a fast-release microsphere and a slow-release microsphere) having different degradation times.

FIG. 3 is a schematic view of another exemplary intravascular delivery system delivering an injectable composition provided herein within a patient's peripheral vasculature.

FIGS. 4-6 are schematic illustrations showing various stages of degradation of the exemplary injectable composition of FIG. 3.

While the embodiments of the systems, devices, and/or methods provided herein are amenable to various modifications and alternative forms, specific embodiments have been shown by way of example in the drawings and are described in detail below. The intention, however, is not to limit this disclosure to the particular embodiments described. On the contrary, the disclosure is intended to cover all modifications, equivalents, and alternatives falling within the scope of the systems, devices, and/or methods as defined by the appended claims.

### DETAILED DESCRIPTION

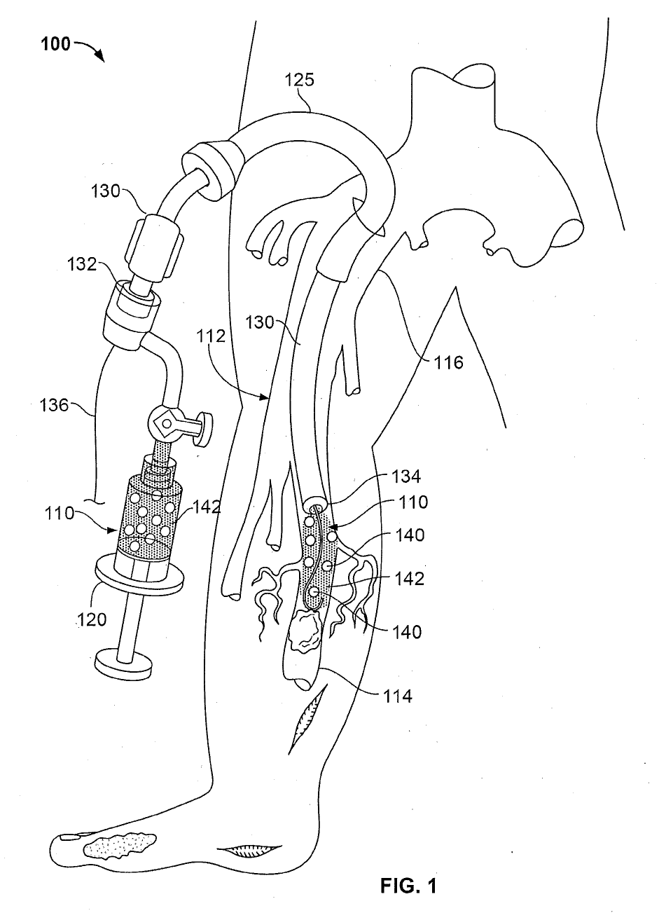

FIG. 1 is a schematic view of an exemplary system 100 for delivering an injectable composition 110 within a patient's peripheral vasculature 112, according to various embodiments of the present disclosure. In some cases, the system 100 can deliver the injectable composition 110 within a patient's arterial or venous vasculature. In some cases, the system 100 can be configured for delivering the injectable composition within a patient's peripheral vasculature, or coronary vasculature. In some cases, system 100 can be configured for delivering the injectable composition 110 within a patient's neurovascular regions. The system 100 provided herein can be used for a wide range of medical applications that deliver the injectable composition 110 within a blood vessel 114, or a blood vascular system (i.e., the circulatory system).

The exemplary device of FIG. 1 includes an injector 120, an introducer sheath 125, a delivery catheter 130, and the injectable composition 110 that is delivered through the catheter 130. The depicted system 100 shows the injector 120 (e.g., a hand-operated or machine-operated syringe) releasably coupled to a proximal end 132 (e.g., a hub or a manifold) of the delivery catheter 130. The depicted delivery catheter includes an elongate tubular shaft extending between the proximal end and a distal tip 134. The delivery catheter 130 can be sized and shaped to be received within a lumen of the introducer sheath 125. The introducer sheath 125 can be inserted into a femoral blood vessel 116 (e.g., femoral artery) at a femoral incision and advanced into the peripheral vasculature towards a lower extremity region. The catheter 130 can be inserted into and extended through the lumen of the sheath 125 into the lower extremity region. The injectable composition 110 provided herein may contain one or more therapeutic agents encapsulated within a microsphere 140 (which can also be referred to as a microbead), or a plurality of microspheres 140. The injectable composition 110 may be supplied by the injector 120 to the catheter 130 and delivered through a lumen of the catheter 130 to the vasculature, and released into the bloodstream at the distal tip 134 of the catheter 130.

In use, the delivery catheter 130 for delivering the injectable composition 110 can be introduced into the patient by a medical practitioner in a cardiac catheterization lab using fluoroscopy. The distal tip 134 of the catheter 130 can be guided to the desired location within the patient's vasculature by advancing the catheter over a guidewire 136. The injector 120 may be filled with the injectable composition 110 by the medical practitioner during the medical procedure, or pre-filled by a company, such as a manufacturer or a distributor. The injector 120 can be connected to the catheter 130, by a connector, such as a luer-fitting (as shown in FIG. 1) or tapered tip (not shown), to the catheter 130. Once the distal tip 134 of the catheter 130 is positioned at the desired location, the injector 120 can be actuated by the medical practitioner or a machine. The injectable composition 110 can be released such that the microspheres 140, which each contain the therapeutic agent, disperse within the targeted vasculature region of the patient. The microspheres 140 may degrade and release the therapeutic agent at an approximated predetermined time, or after a minimum predetermined time, which can be controlled by a rate of degradation of polymer membrane (or also referred to as a “shell”) of the microsphere 140.

In various embodiments provided herein, the composition 110 may include various suitable biocompatible carriers and microspheres 140 containing the therapeutic agent, e.g., stem cells. In some cases, the composition 110 can include a suspension of the microspheres 110 in the carrier 142 (or stem cell solution). In some cases, a suitable weight percentage of the components of the composition (e.g., microspheres 140 and carrier 142) yields a solution having a viscosity approximate to the viscosity of blood.

In some cases, the composition 110 includes the microspheres 140 (and contents therein) in an amount ranging from about 10% to about 90% by weight of the composition 110 and the carrier 142 in an amount ranging from about 90% to about 10% by weight of the composition 110. In some cases, the composition 110 includes the microspheres 140 in an amount ranging from about 10% to about 50% by weight of the composition 110 and the carrier 142 in an amount ranging from about 90% to about 50% by weight of the composition 110. In some cases, the composition 110 includes the microspheres 140 in an amount ranging from about 1% to about 20% by weight of the composition 110 and the carrier 142 in an amount ranging from about 99% to about 80% by weight of the composition 110. Preferably, in some cases, the composition 110 includes the microspheres 140 in an amount ranging from about 30% to about 50% by weight of the composition 110 and the carrier in an amount ranging from about 70% to about 50% by weight of the composition 110. More preferably, in some cases, composition 110 includes the microspheres 140 in an amount ranging from about 35% to about 45% by weight of the composition 110 and the carrier in an amount ranging from about 65% to about 55% by weight of the composition 110.

A suitable carrier can include a liquid carrier such as an aqueous-based solution or a non-aqueous (e.g., organic) solution. Exemplary liquid carriers can include, but are not limited to, purified water, distilled water, saline solution, or a serum (e.g., bovine serum or human serum albumin (HSA)).

### Compositions Including Degradable Microspheres

The injectable composition provided herein can include microspheres each including a wall containing a biodegradable polymer membrane, and a therapeutic agent. The polymer membrane encapsulates the therapeutic agent contained within each microsphere. Encapsulation of the therapeutic agent can allow for a controlled release of the therapeutic agent. Encapsulation can also protect the therapeutic agent from premature degradation. Each microsphere optionally encapsulates a suitable carrier (e.g., a liquid carrier) described herein for forming a suspension containing the therapeutic agent. In some cases, the liquid carrier suspending the microspheres and the liquid carrier suspending the therapeutic agent within the microsphere may be substantially equivalent, substantially similar, or different from one another.

Each microsphere 140 can include a range of suitable weight percentages of the polymer membrane and the therapeutic agent. In some cases, the weight percentage of the therapeutic agent can be configured to deliver a suitable volume of therapeutic agent per microsphere. In some cases, the weight percentage of the polymer membrane may be configured to provide the microsphere with suitable structural stability prior to its degradation, as well as a suitable degradation time in which the polymer membrane of the microsphere disintegrates and allows release of the therapeutic agent from the interior of the microsphere. For example, in some cases, each microsphere includes the polymer membrane in an amount ranging from about 2% to about 50% by weight (e.g., from about 5% to about 40%, from about 10% to about 30%, from about 5% to about 20%, from about from about 2% to about 5%, from about 5% to about 10%, from about 10% to about 20%, from about 20% to about 30% by weight, or from about 30% to about 50% by weight) of the microsphere 140. In some cases, each microsphere includes the therapeutic agent in an amount ranging from about 50% to about 98% by weight (e.g., from about 55% to about 95%, from about 60% to about 80%, from about 50% to about 70%, from about 70% to about 80%, from about 80% to about 90%, from about 90% to about 98% by weight) of the microsphere 140.

In some cases, the microsphere 140 can include a range of suitable weight percentages of the polymer membrane, the therapeutic agent, and a carrier (e.g., saline). In some cases, the weight percentages of the therapeutic agent and the carrier are configured to achieve a solution (e.g., suspension) with a viscosity approximate to the viscosity of blood. For example, in some cases, each microsphere includes the therapeutic agent in an amount ranging from about 10% to about 90% by weight of the microsphere 140 and the carrier in an amount ranging from about 90% to about 10% by weight of the microsphere 140. In some cases, the microsphere includes the therapeutic agent in an amount ranging from about 10% to about 50% by weight of the microsphere 140 and the carrier in an amount ranging from about 90% to about 50% by weight of the microsphere 140. In some cases, each microsphere 140 includes the therapeutic agent in an amount ranging from about 1% to about 20% by weight of the microsphere 140 and the carrier in an amount ranging from about 99% to about 80% by weight of the microsphere 140. Preferably, in some cases, each microsphere 140 includes the therapeutic agent in an amount ranging from about 30% to about 50% by weight of the microsphere 140 and the carrier in an amount ranging from about 70% to about 50% by weight of the microsphere 140. More preferably, in some cases, each microsphere 140 includes the therapeutic agent in an amount of about 40% by weight of the microsphere 140 and the carrier in an amount of about 60% by weight of the microsphere 140.

As used herein, microspheres 140 can include various shapes including, but not limited to, a spherical shape, or a cylindrical shape. The microspheres 140 of the injectable composition provided herein can have a range of suitable diameters. For example, in some cases, the diameter of the microspheres 140 can range from about 10 microns to about 5,000 microns (e.g., from about 20 microns to about 2,000 microns, from about 50 microns to about 1,000 microns, from about 100 microns to about 500 microns, from about 200 microns to about 400 microns, from about 10 microns to about 50 microns, from about 20 microns to about 70 microns, from about 50 microns to about 100 microns, from about 70 microns to about 150 microns, from about 100 microns to about 200 microns, from about 150 microns to about 350 microns, from about 200 microns to about 500 microns, from about 500 microns to about 1,000 microns, from about 1,000 microns to about 2,000 microns, from about 2,000 microns to about 5,000 microns, from about 100 microns to about 300 microns, from about 200 microns to about 300 microns, from about 250 microns to about 400 microns, or from about 200 microns to about 5,000 microns) before an implantation. In some cases, the average diameter of the microspheres may be about 10 microns (e.g., about 50 microns, about 100 microns, about 200 microns, about 250 microns, about 300 microns, about 400 microns, about 500 microns, about 1 mm, about 2 mm, or about 5 mm).

The microspheres 140 provided herein can be sized to a range of suitable volumes. In some cases, each microsphere 140 can be sized to contain from about 0.1 ml to about 2 ml volume of fluid (e.g., from about 0.1 ml to about 1.5 ml, from about 0.2 ml to about 1.2 ml, from about 0.3 ml to about 1.0 ml, from about 0.5 ml to about 0.7 ml, from about 0.1 ml to about 0.3 ml, from about 0.3 ml to about 0.5 ml, from about 0.5 ml to about 0.7 ml, from about 0.7 ml to about 1.0 ml, from about 1.0 ml to about 1.5 ml, or from about 1.5 ml to about 2.0 ml).

Each microsphere 140 provided herein can include the polymer membrane wall made of a biodegradable polymer for encapsulating the therapeutic agent. The polymer membrane of the microspheres 140 provided herein can have any suitable thickness. A suitable thickness can be based on one or more factors, for example, a particular therapeutic treatment type, a treatment dosage rate, and the patient's health. In some cases, the thickness of the polymer membrane is proportional to the microsphere diameter. For example, the polymer membrane thickness can range from about 5% to about 20% (e.g., from about 5% to about 15%, from about 10% to about 15%, from about 5% to about 10%, or from about 5% to about 7%) of the microsphere diameter. In some cases, the thickness of the polymer membrane can range from about 0.5 microns to about 1,000 microns (e.g., from about 0.5 microns to about 750 microns, from about 1 microns to about 500 microns, from about 5 microns to about 250 microns, from about 10 microns to about 100 microns, from about 25 microns to about 50 microns, from about 0.5 microns to about 1 micron, from about 1 micron to about 10 microns, from about 10 microns to about 50 microns, from about 50 microns to about 100 microns, from about 100 microns to about 500 microns, or from about 500 microns to about 1,000 microns).

The injectable composition provided herein can include microspheres with a suitable rate of degradation of the polymer membrane to release the therapeutic agent contained therein. The rate of degradation may be varied to achieve a desired degradation time, where the degradation time is an amount of time in which the polymer membrane of the microsphere 140 disintegrates and allows release of the therapeutic agent from the interior of the microsphere 140. In some cases, the degradation time can range from about 5 minutes to about 72 hours (e.g., from about 15 minutes to about 60 hours, from about 30 minutes to about 48 hours, from about 60 minutes to about 24 hours, from about one hour to about 24 hours, from about 2 hours to about 12 hours, from about 3 hours to about 6 hours, from about 5 minutes to about 30 minutes, from about 30 minutes to about 60 minutes, from about 1 hour to about 3 hours, from about 3 hours to about 6 hours, from about 6 hours to about 12 hours, from about 12 hours to about 24 hours, from about 24 hours to about 48 hours, from about 1 hour to about 48 hours, from about 6 hours to about 24 hours, from about 12 hours to about 24 hours, from about 24 hours to about 48 hours, or from about 48 hours to about 72 hours).

The injectable composition provided herein can include microspheres that include a suitable material for the wall containing the biodegradable polymer membrane. In some cases, a suitable biodegradable polymer membrane includes materials susceptible to enzymatic degradation in the patient's blood. Exemplary materials of the biodegradable polymer membrane can include, without limitation, polylactic acid (PLA), polyglycolic acid (PGA), copolymers of PLA and PGA, poly-L-lactide (PLLA), poly-D,L-lactide (PDLA), poly-capralactone (PCL), and combinations thereof.

The injectable composition can contain various suitable therapeutic agents. Exemplary therapeutic agents can include, without limitation, stem cells, adenoviruses, chemotherapeutic agents, immunosuppressants, proteins, nucleic acids, and combinations thereof. Stems cells can be useful for tissue reconstruction, regeneration and/or repair. Exemplary stem cells can include, without limitation, mesenchymal stem cells that are isolated from adult tissue, induced pluripotent stem cells (iPS cells or iPSCs), embryonic stem cells, and combinations thereof. In some cases, the therapeutic agent includes an effective amount of a protein, such as a hematopoietic progenitor cell antigen CD34 that purportedly activates the immune system. In some cases, the therapeutic agent can include additional ingredients, such as a freezing media, for example, for preserving the therapeutic agent.

The injectable compositions provided herein can be used to deliver therapeutic agents for the treatment of various diseases. The injectable compositions provided herein may also deliver therapeutic agents to various locations within the body that include, but are not limited to, the renal system, or the vascular system within brain, heart, or limbs. For example, the injectable compositions provided herein can deliver one or more therapeutic agents to a targeted vascular region and provide a controlled release over time to treating the targeted areas.

### Compositions Including Fast and Slow-Degrading Microspheres

In some cases, the injectable composition provided herein can include a liquid carrier containing a first microsphere (or a first plurality of microspheres), and a second microsphere (or a second plurality of microspheres), wherein the first microsphere has a different composition and/or a different characteristic (e.g., release profile) than the second microsphere. For example, in some cases, the first microsphere can include a polymer membrane having a different degradation rate (e.g., a faster or slower degradation rate) than a polymer membrane of the second microsphere. In some cases, the polymer membranes of the first microspheres can include a different material than the polymer membranes of the second microspheres. In some cases, the polymer membranes of the first and second microspheres can be made of different types of polymers, copolymers that include different monomer units, or copolymers having different ratios of at least two monomers.

In some cases, the polymer membrane of the first microsphere can have a different thickness than the polymer membrane of the second microsphere to produce at least two or more microspheres having different degradation times. In some examples, the polymer membrane of the first microsphere has a thinner wall than the polymer membrane of the second microsphere such that the first microsphere will have a shorter degradation time than the second microsphere. In some examples, the polymer membranes of the first microspheres may be generally thicker than the polymer membranes of the second microspheres such that the first microsphere will have a longer degradation time than the second microsphere. Accordingly, the first microsphere may be configured to release therapeutic agents faster or slower than the second microsphere based on the thickness ratio of the polymer membrane of the first microsphere relative to the second microsphere. For example, in some cases, the ratio of the average wall thickness of the first microspheres relative to the second microspheres can be about 1:1, 1:2, 1:3, 1:4, 1:5, 5:1, 4:1, 3:1, or 2:1. In some cases, the ratio of the average wall thickness of the first microspheres relative to the second microspheres can range from about 1:1 to about 1:5 (e.g., from about 1:1 to about 1:4, from about 1:1 to about 1:3, from about 1:1 to about 1:2, from about 1:2 to about 1:4, from about 1:2 to about 1:3, from about 1:3 to about 1:4, or from about 1:4 to about 1:5). In some cases, the ratio of the average wall thickness of the first microspheres relative to the second microspheres can range from about 5:1 to about 1:1 (e.g., from about 5:1 to about 2:1, from about 5:1 to about 3:1, from about 5:1 to about 4:1, from about 4:1 to about 1:1, from about 4:1 to about 2:1, from about 4:1 to about 3:1, from about 3:1 to about 1:1, or from about 3:1 to about 2:1, or from about 2:1 to about 1:1).

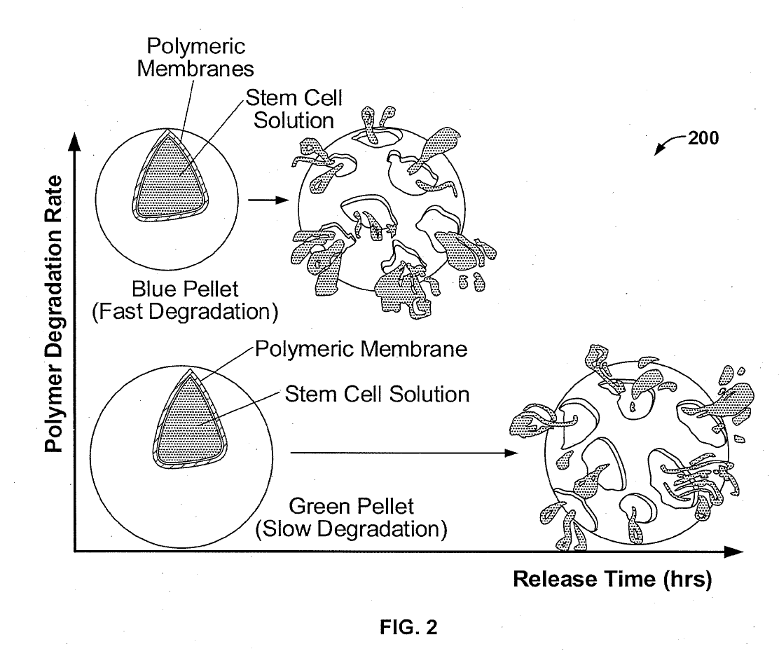

FIG. 2 is a graph 200 illustratively comparing two different exemplary microspheres (e.g., a slow-release microsphere and a fast-release microsphere) that have different degradation times. Certain embodiments of the compositions provided herein include at least two different types of microspheres, where each microsphere type has a different degradation rate. As discussed herein, compositions can include fast-release microspheres and slow-release microspheres, in which the wall of the fast-release microspheres has a faster average degradation rate or a shorter time span for degrading, as compared to the average degradation rate or degradation time of the wall of the slow-release microspheres. As depicted in the figures, an exemplary fast-release microsphere can be associated to a low release time and a high degradation rate of the polymer (outer) membrane while an exemplary slow-release microsphere is associated with a high release time and a low degradation rate of the polymer membrane. The benefit of the injectable composition with variable-release microspheres includes allowing a medical practitioner to deliver to the patient during a single intravascular procedure a treatment that provides multiple therapeutic agent exposures over a given period of time.

The injectable compositions provided herein can be made with a suitable fast-release degradation time and a suitable slow-release degradation time. For example, in some cases, the fast-release microspheres can degrade within 24 hours (e.g., within 1 hour, 3 hours, 6 hours, 9 hours, 12 hours, or 24 hours) and the slow-release microspheres can degrade between 24 hours and 72 hours (e.g., 25 hours, 30 hours, 40 hours, 48 hours, 54 hours, 60 hours, or 72 hours). In some cases, the fast-release microspheres can degrade within 1 week (e.g., within 1 day, 2 days, 3 days, 4 days, 5 day, 6 days, or 7 days) and the slow-release microspheres can degrade from about 1 week to about 1 month (1 week, 2 weeks, 3 weeks, 4 weeks, 1 month). In some cases, the slow-release microspheres can have a degradation time that is about 1.1, about 1.2, about 1.5, about 2, about 3, about 4, about 5, about 10, about 20, or greater than 20 times greater than the degradation time of the fast-release microspheres. In some cases, slow-release microspheres degrade about 1 hour to about 1 month (e.g., about 1 hour, about 2 hours, about 3 hours, about 6 hours, about 12 hours, about 24 hours, about 48 hours, about 54 hours, about 60 hours, about 72 hours) after the fast-release microspheres have degraded.

Certain embodiments of the injectable composition provided herein contain at least two or more microspheres containing different intra-microsphere materials. For example, in some cases, the injectable composition provided herein can include first microspheres containing a different therapeutic agent than the therapeutic agent of the second microspheres. In some cases, the first and second microspheres can contain different concentrations of the same therapeutic agent.

### Compositions Including Multi-Encapsulated Microspheres

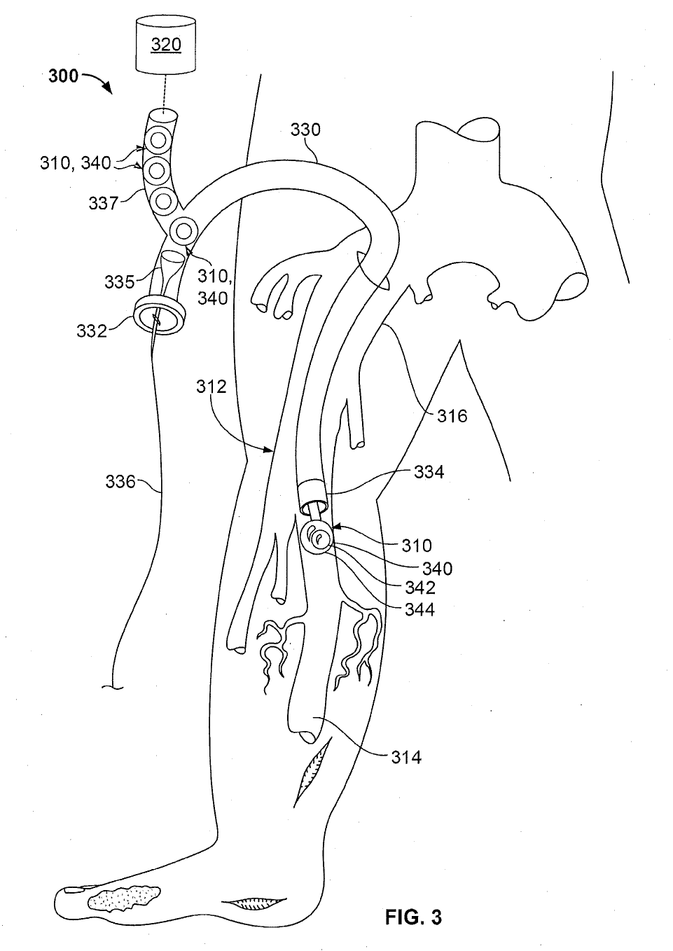

FIG. 3 is a schematic view of another exemplary intravascular delivery system 300 delivering an injectable composition 310 within a patient's peripheral vasculature 312. The exemplary device of FIG. 1 includes a supply source 320, a delivery catheter 330, an optional dispensing device 335, and the injectable composition 310 being delivered by the catheter 330. The catheter 330 may be positioned within the anatomy using a guidewire 336 and optionally introduced with an introducer sheath (not shown). The depicted system 300 shows the supply source 320 coupled to and in fluid connection with a side port 337 of the catheter 330. The delivery catheter 330 can be inserted into a femoral blood vessel 316 (e.g., femoral artery) at a femoral incision and extended within the peripheral vasculature toward a patient's lower extremity. The dispensing device 335 can be inserted through an opening at a proximal end 332 (e.g., a hub or a manifold) of the delivery catheter 330, and optionally includes a cup-shaped tip 335. As depicted, an injectable composition 310 provided herein is provided by the supply source 320, delivered through the delivery catheter 330, and released into the patient's vasculature 312 at a distal tip 334 of the catheter 330. The injectable composition 310 contains a therapeutic agent encapsulated within one or more multi-encapsulated microspheres 340. As shown in FIG. 3, the multi-encapsulated microspheres 340 include at least one (smaller) microsphere 342 encapsulated within another (larger) microsphere 344. In some cases, the injectable composition can be transported through the catheter 330 by advancing the dispensing device 335 such that the cup-shaped tip contacts at least a portion of one or more multi-encapsulated microspheres and pushes the one or more microspheres 340 through the lumen of the catheter 330 and to a targeted delivery site.

In some embodiments, the device provided herein contains a composition that includes the multi-encapsulated microspheres 340, in which at least one (smaller) microsphere 342 is encapsulated within another (larger) microsphere 344, to provide the benefit of delivering a therapeutic agent into the vasculature at different times, or to release the agents at different locations within the vasculature. Certain embodiments of the injectable composition 310 provided herein include multi-encapsulated microspheres 340 that allow for a controlled, prolonged delivery of a therapeutic agent. The outer microsphere 344 can include a wall containing a biodegradable polymer membrane that envelopes a therapeutic agent optionally suspended in a carrier, and at least one smaller (inner) microsphere 342. The polymer membrane of the outer microsphere 344 encapsulates the therapeutic agent and the inner microsphere 342 contained within the outer microsphere 344. Encapsulation of the therapeutic agent within the outer microsphere 344 can allow for the controlled release of the therapeutic agent and inner microsphere 342 contained therein. The inner microsphere 342 also includes a wall containing a biodegradable polymer membrane, and a therapeutic agent optionally suspended in a carrier. The inner microsphere 342 encapsulates the therapeutic agent and allows for a prolonged, staged release of a therapeutic agent.

In some cases, the outer microsphere can include more than one inner microsphere (e.g., two or more microspheres, three or more microspheres, four or more microspheres, five or more microspheres, ten or more microspheres, twenty or more microspheres, thirty or more microspheres, forty or more microspheres, or fifty or more microspheres).

Some embodiments of the injectable composition 310 provided herein include multi-encapsulated microspheres (not shown). For example, in some cases, the injectable composition 310 provided herein can include two inner microspheres; a first inner microsphere that encapsulates a second inner microsphere. In some cases, the injectable composition provided herein can include three, four, five, or more than five inner microspheres, where each microsphere is encapsulated within another microsphere with exception of the smallest microsphere.

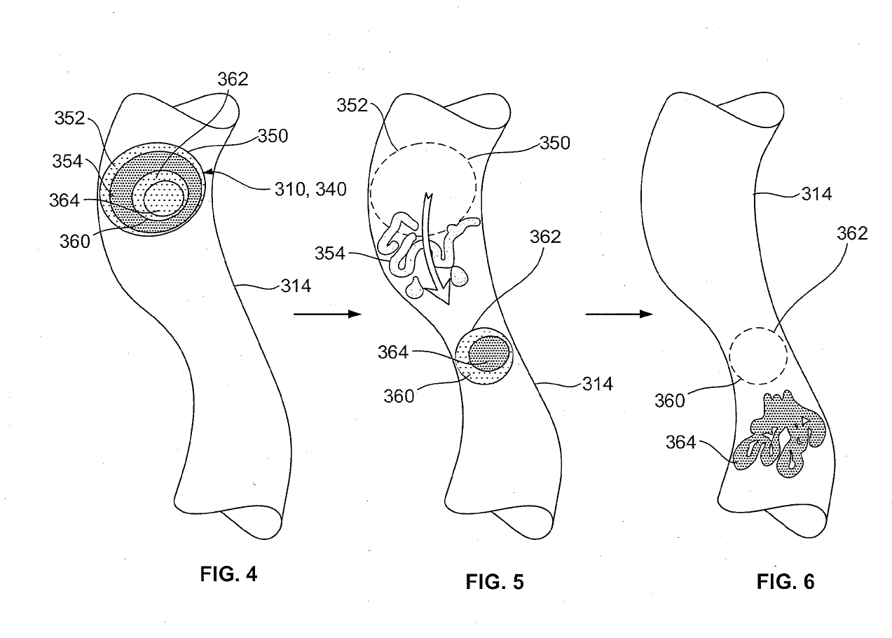

FIGS. 4-6 are schematic illustrations showing various stages of degradation of an exemplary multi-encapsulated microsphere 340 of the injectable composition 310 shown in FIG. 3 within a blood vessel 314. Referring to FIG. 4, the microsphere 340 of the injectable composition 310 provided herein contains the outer microsphere 350. The outer microspheres 350 include the wall containing the biodegradable polymer membrane 352 encapsulating the therapeutic agent 354 (and optionally a carrier) and the inner microsphere 360. After being injected into the vasculature, the outer microsphere 350 can travel from a larger blood vessel at the injection location into a smaller vessel within the vasculature. In some cases, the outer microsphere 350 can eventually become lodged within a blood vessel 314 having a luminal diameter comparable to the outer diameter of the outer microsphere 350, as shown in FIG. 4. Accordingly, the outer diameter of the outer microsphere 350 can be predetermined to target a particular blood vessel size, or a size range, for releasing the therapeutic agent 354.

Referring to FIG. 5, the polymer membrane 352 of the outer microsphere 350 degrades over time and eventually releases its internal contents (e.g., the therapeutic agent 354 and optional carrier) into the blood vessel 314. Once the polymer membrane 350 has degraded, the therapeutic agent 354 previously contained between the polymer membranes 352, 362 of the outer and inner microspheres 350, 360 releases into the vasculature. Degradation of the outer microsphere 350 also releases the inner microsphere 360. The inner microsphere 360 has a smaller diameter than the outer microsphere 350, thus the inner microsphere 360 is allowed to travel to smaller blood vessels 314 within the vasculature. Accordingly, the multi-encapsulated microspheres 340 provide the benefit of targeting two or more blood vessel sizes for releasing the therapeutic agent 354. Another benefit of the multi-encapsulated microsphere 340 includes providing the patient with multiple exposures of the therapeutic agent 354 because the inner microsphere 360 does not begin to degrade until the outer microsphere 350 has sufficiently degraded and allows the inner microsphere 360 to become exposed to the blood and the proteins therein.

Referring to FIG. 6, the inner microsphere 360 can become eventually lodged within a smaller blood vessel before releasing the therapeutic agent 364 contained therein. The polymer membrane 362 of the inner microsphere 360 degrades over time and eventually releases the therapeutic agent 364 contained within the inner microsphere 360 into the vasculature. Due to the sequenced release of the outer and inner microspheres 350, 360, the advantages of the injectable compositions 340 provided herein include providing a controlled, prolonged therapeutic agent release within the patient's body and targeted (vessel size) release at various locations along the vasculature.

### Methods of Manufacturing

There are a number of processes available for manufacturing microspheres of the injectable compositions provided herein. Exemplary processes, which can depend on particular materials used, can include, but are not limited to, phase separation or precipitation processes, emulsion/solvent evaporation processes, spraying processes, extrusion processes, injection molding processes, injection or microinjection processes, sphere blowing processes, electrospinning processes, 3D printing, and combinations thereof. Processes involving injections for filling the microspheres provided herein, can optionally include a heating step for sealing one or more injection site(s).

In some cases, the microspheres of the injectable compositions provided herein can be made by using a microparticle preparation technique, as described in the following reference: Makadia, H. K., and Siegel, S. J. Poly Lactic-co-Glycolic Acid (PLGA) as Biodegradable Controlled Drug Delivery Carrier, Polymers (Basel); 3(3): 1377-1397; 2011.

Some exemplary manufacturing processes for creating microspheres of the injectable composition provided herein include using an immiscible solution process, or emulsion and solvent evaporation process. For example, microspheres can be made using a solvent evaporation and solvent extraction process as described by Jain R. A., The manufacturing techniques of various drug loaded biodegradable poly(lactide-co-glycolide) (PLGA) devices. Biomaterials; 21:2475-2490; 2000 (“the Jain reference”).

In some cases, the immiscible solution process can include formulating a solution to form the polymer membrane (i.e., shell). In some cases, the immiscible solution process can include formulating a solution to form the polymer membrane (i.e., shell). Exemplary solutions may be formed by dissolving a desired amount of a polymer solution (e.g., PLGA and/or PLLA in a solvent, such as dimethylformamide (DMF)). An immiscible therapeutic agent may be added to the polymer membrane solution to form spheres within the solution. Any excess solution can subsequently be removed to yield microspheres that include the therapeutic agent coated in a solidified polymer membrane made from the shell solution.

Certain exemplary manufacturing processes for creating microspheres of the injectable composition provided herein can use a “bubble-forming” method. For example, a thin layer of a soft biodegradable material (e.g., a polymer such as PLGA or PLLA) may be injected with a therapeutic agent using, for example, a needle injector, to form a pocket (or bubble) filled with the therapeutic agent inside the layer of the biodegradable material. In some cases, the therapeutic agent can be added until the layer of the biodegradable stretches to form walls of the microspheres described herein. The injection site(s) (i.e., puncture sites) of the microsphere can be sealed by applying heat at a temperature near or at the glass transition temperature, or the melt temperature, of the layer of the biodegradable material to form a microsphere that encapsulates the therapeutic agent within the layer of the biodegradable material.

Some exemplary manufacturing processes for creating microspheres of the injectable composition provided herein may include using an extrusion method. In some cases, a material (e.g., PLGA) may be extruded through an extrusion dye to form a microtube or a micropellet. Each microtube or micropellet can be filled with a therapeutic agent by, for example, injecting the therapeutic agent into a center portion of the microtube or micropellet with a needle injector. In some cases, injected microtubes may be cut and sealed simultaneously to form cylinder-shaped vesicles that are filled with the therapeutic agent.

In some cases, the microspheres provided herein can be made by using a spraying process. For example, a therapeutic agent may be dispersed as droplets on a polymer film (e.g., a PLGA film). After droplets of the therapeutic agent have been placed onto the polymer film, a polymer membrane solution can be sprayed over the droplets to form microspheres that include the polymer membrane which encapsulates the droplets of the therapeutic agent. In some cases, the spray drying process as well as other types of microsphere forming processes (e.g., double emulsion process or a phase separation process) as described by the Jain reference, may be used to create microspheres provided herein. See id. at 2478-2480.

Some embodiments of the microspheres provided can be made using a molding process and an injection process. For example, in some cases, a mold can be injected with a suitable polymer material provided herein to create the wall of the microsphere. Once the microsphere wall has been completed, the interior hollow region may be filled with a therapeutic agent using an injector that pierces the wall in one or multiple locations. The pierced locations can optionally be sealed by using a heating process that allows the polymer membrane wall to reflow and fill any punctures.

In some cases, 3D printing can be used to form the microspheres provided herein. For example, a first nozzle of a 3D printer may be used to form the wall of a first microsphere while a second nozzle of the 3D printer fills the interior cavity of the first microsphere with a therapeutic agent. Optionally, the 3D printer can be used to form the wall and fill the interior cavity of a second microsphere within the first microsphere. In some cases, the 3D printer may be used to form wall of a hollow (empty) microsphere such that the interior hollow region can be filled with a therapeutic agent by an injector that pierces the wall in one or more locations. An optional heating process may be applied following the injection process to seal any puncture sites.

Some embodiments of the microspheres can be made using an electrospinning process. For example, one or more droplets of a therapeutic agent can be placed on a base plate (or flat surface) at a desired contact angle (e.g., at a 147° angle relative to a horizontal plane) to promote movement of the droplet. An electrospun material can be disposed onto the droplet as it rolls along the flat surface to form a microsphere in which the electrospun material forms the polymer wall that encapsulates the therapeutic droplet.

It should be understood that one or more design features of the embodiments provided herein can be combined with other features of other embodiments provided herein. In effect, hybrid designs that combine various features from two or more of the device designs provided herein can be created, and are within the scope of this disclosure.

While this specification contains many specific implementation details, these should not be construed as limitations on the scope of the disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition to being directed to the teachings described above and claimed below, systems, devices, and methods having different combinations of the features described above and claimed below are contemplated. As such, the description is also directed to other devices and/or methods having any other possible combination of the dependent features claimed below.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the present disclosure, to the full extent indicated by the broad, general meaning of the terms in which the appended claims are expressed. To the extent that these various modifications do not depart from the spirit and scope of the appended claims, they are intended to be encompassed therein. All references, publications, and patents referred to herein, including the figures and drawings included therewith, are incorporated by reference in their entirety.

What is claimed is:

1. An injectable composition for intravascular delivery of a therapeutic agent, the injectable composition comprising:
one or more first microspheres containing a first therapeutic agent, each first microsphere including a wall comprising a biodegradable polymer that encapsulates the first therapeutic agent;
one or more second microspheres containing a second therapeutic agent, each second microsphere including a wall comprising the biodegradable polymer that encapsulates the second therapeutic agent; and
a liquid carrier.

2. The injectable composition of claim 1, wherein the first therapeutic agent, the second therapeutic agent, or both, are selected from a group consisting of stem cells, adenoviruses, chemotherapeutic agents, immunosuppressants, proteins, nucleic acids, or a combination thereof.

3. The injectable composition of claim 1, wherein the injectable composition comprises the first and second microspheres in an amount from about 10% to about 50% by weight of the injectable composition and the liquid carrier in an amount from about 90% to about 50% by weight of the injectable composition.

4. The injectable composition of claim 1, wherein the injectable composition comprises the first and second microspheres in an amount ranging from about 35% to about 45% by weight of the injectable composition and the carrier in an amount of about 65% to about 55% by weight of the injectable composition.

5. The injectable composition of claim 1, wherein the first microspheres, the second microspheres, or both, are suspended in the liquid carrier.

6. The injectable composition of claim 1, wherein one or more first microspheres is disposed within at least one second microsphere.

7. The injectable composition of claim 1, wherein the liquid carrier comprises purified water, distilled water, saline solution, or a serum.

8. The injectable composition of claim 1, wherein the wall of the first microspheres has a faster degradation rate than the wall of the second microspheres.

9. The injectable composition of claim 1, wherein an average wall thickness of the first microspheres is greater than an average wall thickness of the second microspheres.

10. The injectable composition of claim 1, wherein the walls of the first microspheres, the second microspheres, or both, comprise a nonporous polymer layer.

11. The injectable composition of claim 1, wherein the first microspheres further encapsulate at least a portion of the liquid carrier such that the first therapeutic agent is suspended within the liquid carrier inside the first microspheres.

12. The injectable composition of claim 1, wherein the first microspheres, the second microspheres, or both, have diameters ranging from about 0.2 millimeters to about 5.0 millimeters.

13. The injectable composition of claim 1, wherein the biodegradable polymer comprises polylactic acid (PLA), polyglycolic acid (PGA), a copolymer of PLA and PGA, poly-L-lactide (PLLA), poly-D,L-lactide (PDLA), poly-capralactone (PCL), or a combination thereof.

14. A system for intravascular delivery of an injectable composition, the system comprising:
a catheter comprising a proximal end, a distal end, and an elongate tubular shaft defining a lumen;
a source of the injectable composition in fluid communication with the lumen of the catheter, the injectable composition comprising:
at least one first microsphere containing a first therapeutic agent, the first microsphere including a wall comprising a first biodegradable polymer that encapsulates the first therapeutic agent;
at least one second microsphere containing a second therapeutic agent, the second microsphere including a wall comprising a second biodegradable polymer that encapsulates the second therapeutic agent; and
a liquid carrier containing the first microsphere and the second microsphere; and
a transporting element for transporting the injectable composition through the catheter.

15. The system of claim 14, wherein the transporting element comprises an injector for applying an injection pressure to transport the injectable composition through the catheter.

16. The system of claim 14, wherein the transporting element comprises a dispensing device that includes a cup-shaped tip for contacting at least a portion of one or more microspheres and pushing the one or more microspheres through the catheter.

17. A method of manufacturing an injectable composition, the method comprising:
forming a polymer membrane comprising PLGA or PLLA;
adding a therapeutic agent; and
encapsulating the therapeutic agent within the polymer membrane.

18. The method of claim 17, wherein the forming and encapsulating comprise adding an immiscible therapeutic agent into a polymeric solution comprising dimethylformamide and PLGA or PLLA.

19. The method of claim 17, wherein the forming comprises extruding a thin film or microtube or micropellet comprising PLGA or PLLA.

20. The method of claim 17, wherein the encapsulating comprises injecting the therapeutic agent into a film such that the film stretches to form a microsphere.
